(19) United States
(12) Patent Application Publication
(10) Pub. No.: US 2004/0039626 A1
(43) Pub. Date: Feb. 26, 2004

(54) System and method for tracking appointment data

(76) Inventor: Dirk Voorhees, Houston, TX (US)

Correspondence Address: WILSON SONSINI GOODRICH & ROSATI, 650 PAGE MILL ROAD, PALO ALTO, CA, 943041050

(21) Appl. No.: 10/381,968

(22) PCT Filed: Sep. 26, 2001
(86) PCT No.: PCT/US01/30269

(51) Int. Cl. G06F 17/60

(52) U.S. Cl. 705/9; 705/2; 705/5

(57) ABSTRACT

A system and method for tracking appointment data are introduced that combat the many drawbacks associated with conventional systems. A particular embodiment of a system incorporating teachings of the present disclosure includes a clinic engine operable to allow an appointment target to identify a first block of time as available to meet time and an appointment engine operable to make available at least a portion of the first block of time to an appointment seeker in response to an appointment request. The system may also include an appointment seeker interface that allows the appointment seeker to schedule a first appointment during the first block of time and a notification engine operable to notify the appointment target of the first appointment.

### FIELD OF THE INVENTION

The present invention relates generally to scheduling applications and, more specifically, to a system and method for tracking appointment data.

### BACKGROUND OF THE INVENTION

Advances in internetworking technology and the development of more intuitive user interfaces provide enterprises with an opportunity to shift labor intensive business processes like scheduling appointments to more automated solutions. Unfortunately, the currently available techniques for accomplishing this objective are inflexible and difficult to use

Conventional options for tracking appointment data, including pen and paper (“PnP”) solutions and commercially available software solutions, have substantial disadvantages. PnP solutions require an enterprise employee to check appointment availability by referring to one or several calendars. The employee must then relay the available appointment options to the party seeking the appointment and schedule the party's appointment. The process can be exceedingly slow and unreliable. Moreover, PnP solutions require the presence of an individual, which means an enterprise seeking to have twenty-four hour a day coverage will need to employ at least three shifts of people—making PnP solutions costly.

In recent years, some enterprises have replaced various aspects of the PnP solution with software applications. For example, an employee consulting calendars in search of an available appointment time may be able to do so via a personal computer (“PC”). This may improve the overall efficiency and accuracy of the scheduling system if the calendars being checked are up to date and accurate. Unfortunately, because there may be several layers of abstraction in a large enterprise, individual calendars are not always accurate.

In addition, several complex software scheduling solutions require a high level of system similarity between the various users. Basically, everyone should be running the same software. In addition, conventional software solutions tend to be Email based. For example, if an end user of a conventional software system wants to schedule an appointment with a second user, the end user makes a request and an Email is generated and sent to the second user. The Email request may, in more complex systems, allow the second user to click on a link in the Email and accept or decline the appointment. As such, the party seeking to schedule an appointment cannot be sure the appointment is firm until the second user reads the appropriate Email and accepts the appointment. In many time sensitive arenas, such a delay is unacceptable.

Accordingly, there is a need for improved systems and methods for tracking appointment data.

### SUMMARY OF THE INVENTION

In accordance with the teachings of the present invention, a system and method for tracking appointment data is provided. A particular embodiment of a system incorporating teachings of the present disclosure includes a clinic engine operable to allow an appointment target to identify a first block of time as available-to-meet time and an appointment engine operable to make available at least a portion of the first block of time to an appointment seeker in response to an appointment request. The system may also include an appointment seeker interface that allows the appointment seeker to schedule a first appointment during the first block of time and a notification engine operable to notify the appointment target of the first appointment. In other embodiments, the clinic engine may be further operable to allow the appointment target to identify allowable locations for the first appointment. In addition, the clinic engine may be further operable to allow the appointment target to identify an allowable duration for the first appointment and an allowable class of user for given blocks of time. The above example systems incorporating teachings of the present invention may include various software engines. An engine may include, for example, computer operations running in separate computing platforms or the same computing platforms. The computer operations may be written to be object-oriented and may make use of different languages including, for example, third generation languages like Java, Visual Basic, C++, and PL/S. In some embodiments, engines may be modular and identifiable as separate discrete blocks of code. In other embodiments, engines may be included within and integrated into one or more larger blocks of code.

In accordance with a further embodiment, a method incorporating teachings of the present disclosure may include maintaining a data store comprising information relating to an appointment schedule of an appointment target. The appointment target may be, for example, an individual, a group, or an organization, with whom another individual, group, or organization would like to meet. The method embodiment described above for tracking appointment information may include identifying an appointment opportunity for the appointment target as open for scheduling or available-to-meet and receiving a request to schedule an appointment during the appointment opportunity. In some embodiments the request may be granted in real time and without further consultation with the appointment target though the appointment target may be subsequently notified of the granted request.

Systems and methods incorporating teachings of the present disclosure offer significant benefits over conventional and currently available systems and methods. For example, the present teachings provide for a solution that is both fast and efficient. An appointment target, perhaps a doctor, an automobile mechanic, a barber, or an executive, can preemptively establish their availability and publish the availability to appointment seekers. As such, an appointment seeker, for example a patient or an individual in need of a hair cut, may select an appointment and, in some embodiments, receive real time confirmation of the appointment. The appointment seeker may no longer be forced to wait until the target can respond to a request for a meeting.

Moreover, some embodiments may be implemented as software engines on local or distributed computing platforms and networks. In preferred embodiments, these systems may be available twenty-four hours a day and readily accessible via a large computer network such as the Internet or enterprise intranets. In some embodiments, users may access the system using communication protocols like TCP/IP.

Additional advantages may be apparent from reference to the attached figures and their description.

### BRIEF DESCRIPTION OF THE DRAWINGS

A more complete understanding of the present embodiments may be acquired by referring to the following description taken in conjunction with the accompanying drawings, in which like reference numbers indicate like features, and wherein:

FIG. 1 depicts a block diagram of one embodiment of a system incorporating teachings of the present disclosure.

FIG. 2 depicts a block diagram of another embodiment of a system incorporating teachings of the present disclosure.

FIG. 3 shows a state transition diagram that illustrates operation and use of a system embodiment incorporating teachings of the present disclosure.

FIG. 4 shows one embodiment of a graphical user interface presented to a system user at log in.

FIG. 5 shows one embodiment of a graphical user interface presented to a system administrator.

FIG. 6 shows one embodiment of a graphical user interface presented to an appointment target identifying blocks of available to meet time.

FIG. 7 shows one embodiment of a graphical user interface presented to an appointment seeker viewing a calendar of a specific target's available to meet time.

FIG. 8 shows one embodiment of a graphical user interface presented to an appointment seeker searching for and scheduling an appointment.

FIG. 9 shows one embodiment of a graphical user interface presented to an appointment seeker depicting the seeker's scheduled appointments.

### DETAILED DESCRIPTION OF THE DRAWINGS

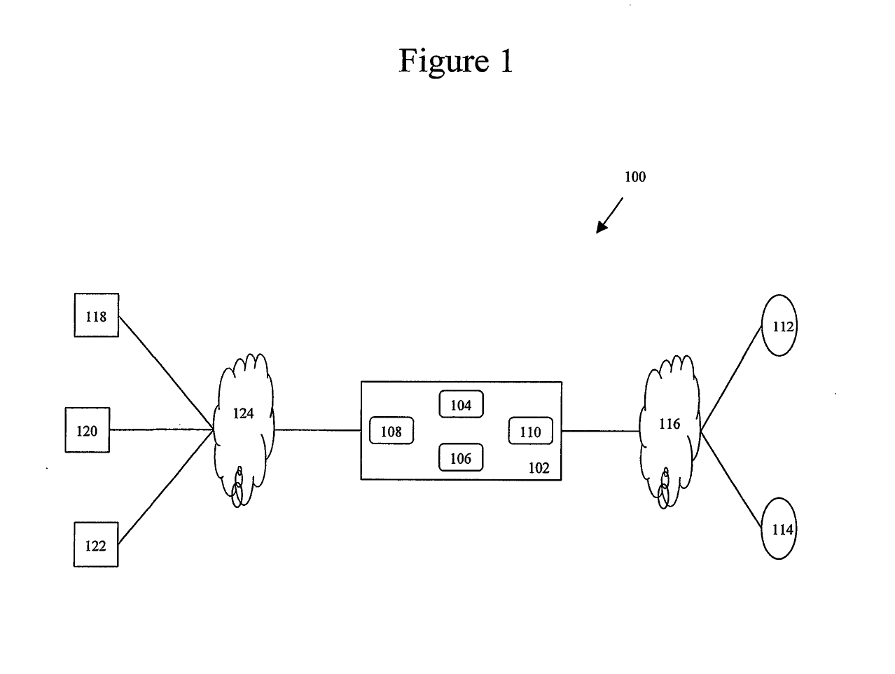

Referring to FIG. 1, a system 100 is depicted. As depicted, system 100 may include a server 102 executing various software engines. For example, system 100 may include a clinic engine 104, an appointment engine 106, an interface engine 108, and a notification engine 110. An engine may include, for example, computer operations running in separate computing platforms or the same computing platform. In some embodiments, the computer operations may be written to be object-oriented and may make use of different languages including, for example, third generation languages like Java, Visual Basic, C++, and PL/S.

In operation, system 100 may help an appointment seeker, such as a patient, receive real time and web based confirmation of an appointment with an appointment target, such as a doctor. The system may also provide the functionality an organization, enterprise, or entity needs to manage other processes that are triggered when an appointment occurs (e.g., electronic payment claims, on-line prescriptions, on-line diagnosis, etc.). In preferred embodiments, the system may be secure, and the privacy of target and seeker information protected. System 100 may be Internet based. System 100 may also be embedded in an organization's existing intranet site or set up on an extranet to give an organization real-time scheduling functionality.

In use, system 100 may allow an appointment target 112, 114 to access server 102 through a communication network 116, such as the Internet, using communication protocols like TCP/IP. Once accessed, system 100 may allow an appointment target 112, 114 to establish and publish an availability summary. For example, clinic engine 104 may allow a doctor to create, modify, or delete various types of information about the doctor's schedule. For example, clinic engine 104 may allow a doctor to list his or her specialty, the facility or facilities where the doctor will meet patients, the appointment type (e.g., standard consultation, special consultation, or surgery), the appointment interval, or the time and date range the doctor is available to meet. Clinic engine 104 may also allow for additional criteria to be input.

Once a clinic has been established using clinic engine 104, an appointment seeker 118, 120, 122 may access interface 108 through a communication network 124. Appointment seeker 118, 120, 122 may search for an individual or type of target using appointment engine 106. Appointment engine 106 may compare a seeker's request against the known availability of targets that have established a clinic and present a seeker with targets who are available to meet. In preferred embodiments, a seeker may select a target and receive real time confirmation of the appointment and its time, place, etc. Because a target may preemptively state when he or she is available, an appointment may be confirmed quickly and notification engine 110 may initiate the sending of a meeting notice to a target.

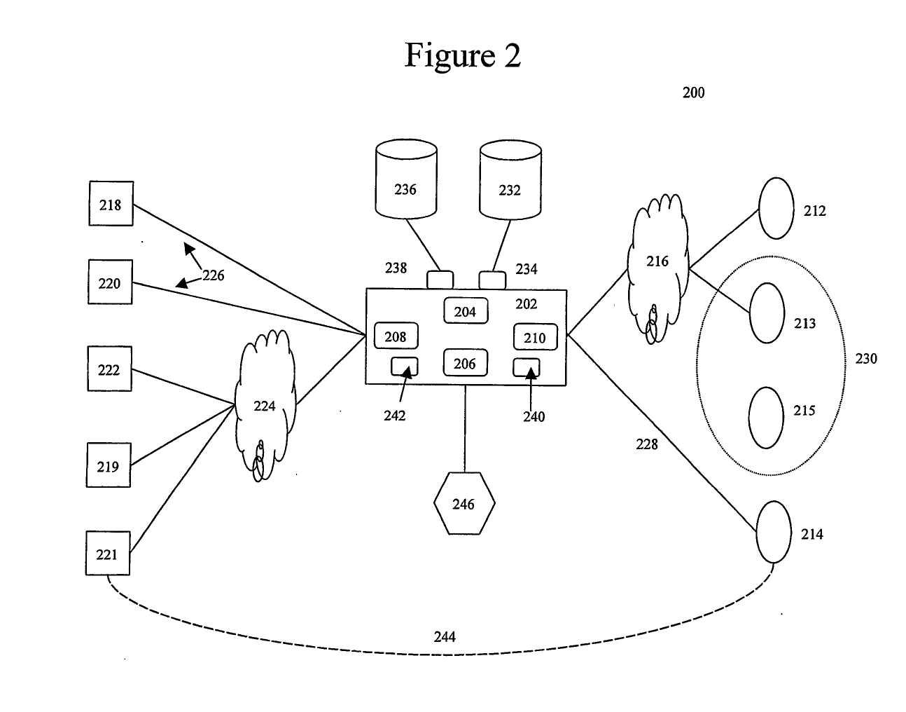

In some applications (e.g., medical applications), a target may want to have some information about the seeker before the meeting. As such, some embodiments of system 100 may include a form or chart to be filled out by the seeker. In preferred embodiments, a seeker may be a registered user or have used system 100 before and his or her information may be stored in a data store and prepopulated into the form or chart. As such, other functionality, such as the functionality depicted in FIG. 2, may be added to system 100.

In the system of FIG. 2, additional functionality has been added. System 200 may include a clinic engine 204, an appointment engine 206, an interface engine 208, and a notification engine 210. In addition, system 200 may include targets 212, 213, 214, 215, some with access through communication network 216, and seekers 218, 219, 200, 221, 222, some with access through communication network 224. But, in addition, system 200 may allow for direct access links 226 between seekers and server 202 and a direct access link 228 between target 214 and server 202.

Moreover, as discussed with system 100, a system incorporating teachings of the present disclosure may seek to notify a target or group of targets 230 of a scheduled meeting. The notification may involve electronic mail and may involve accessing an enterprise Email server 232 via an email interface 234. A system incorporating teachings of the present disclosure may also seek to prepopulate forms with data from a legacy system 236 across a legacy interface 238. Accessing existing data stores and enterprise servers may allow system 200 to operate very efficiently and without the complete duplication of information already available.

Other advantages of system 200 may include a calendar engine 240 operable to generate a calendar that may make selecting an appointment time and date simpler. The calendar generated may be presented to a user when the user is either seeking to make time available or schedule a meeting. Similarly, system 200 may include an update engine 242 that recognizes when a seeker has scheduled an appointment with a target and automatically updates the targets calendar to indicate that the scheduled time is no longer available for appointment. In some embodiments, update engine 242 may also recognize when information located within legacy system 236 has changed and initiate an updating of the information located within legacy system 236 to reflect the change. Other engines may also be present in system 200. For example, some users may want to offer survey capabilities. A system administrator may want to develop and or present a survey to an appointment seeker to determine any number of things, such as, usability of system 200, availability of targets, professionalism of targets, etc. In some embodiments, a survey engine may allow almost anyone to create, distribute, and/or respond to a survey in real time.

Use of system 200 may also involve uses more complex than scheduling a meeting or creating and responding to a survey. On occasion a seeker may need to meet a specific target and may not be able to do so face-to-face. For example, in telemedicine applications, a remotely located patient may need to meet with a specialist. If a person on an aircraft carrier or submarine needs to meet with a dermatologist and a dermatologist is not on ship, the patient may need to virtually meet with the doctor. As such, system 200 may need to recognize that a seeker has scheduled a virtual meeting and initiate provisioning of a virtual meeting link 244 such as a teleconference or videoconference link. In some embodiments, system 200 may allow a seeker to forward information to a target in lieu of or in addition to establishing a telecommunication link. For example, a seeker scheduling a virtual dermatologist meeting might take digital photographs of an infected skin area and system 200 may allow for attachment of the image files to an appointment notice being sent to the dermatologist target.

As may be apparent from the preceding example, system 200 may involve significant complexities. As such, system 200 may include an administrator console 246. Administrator console 246 may take several forms. For example, administrator console 246 may be a separate PC directly linked to server 202, a software engine, or a specific type of user. Administrator console 246 may allow for the provisioning of rights or access within system 200. For example, certain targets may not be permitted to schedule virtual meetings or group 230 may not be allowed to include certain facility locations in clinics or schedules it establishes.

Through administrator console 246, entities of system 200, whether system, software, or information instances, may be managed (either directly or indirectly) using a graphical user-interface. A system administrator may add particular entities to the system's scheduling domain, based on the initial requirements of the enterprise applications being made available. As the scheduling domain evolves, the administrator may manage the entities by modifying ones that currently exist, adding more, or removing ones that are no longer necessary.

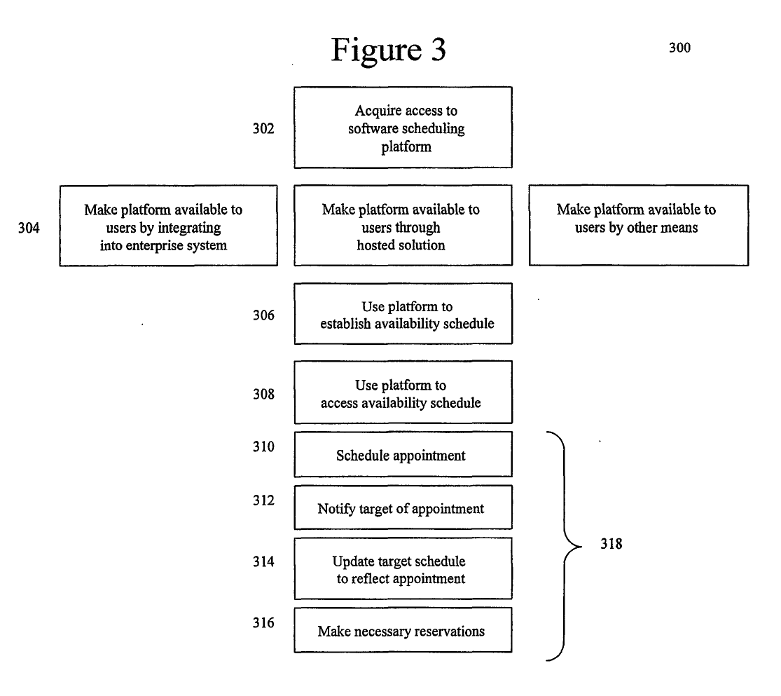

Some applications of a system incorporating teachings of the present disclosure may be better understood by reference to the flow chart of FIG. 3. Depicted method 300 of FIG. 3 may include several stages. For example, at stage 302 an enterprise or entity may acquire access to a scheduling platform incorporating teachings of the present disclosure. The platform may support a system like system 200 of FIG. 2. The enterprise or entity may choose any or all of several techniques for maling the platform available to users. For example, stage 304 depicts an integration option whereby the platform is incorporated into an existing system, a hosting or application service provider (“ASP”) model whereby the platform executes on a remote server and may support more than one enterprise, and a catch all option, which may include a stand alone extranet or intranet option. Depending on an entity's needs, some or all of these access techniques may be employed.

In practice, before access can be granted and functionality and interface appearance can be designed and developed, operational requirements or guidelines may need to be established. For example, guidelines may be established that articulate goals and metrics for tracking the appointment data of one or more entities. For example, an insurance company may be interested in tracking appointment information to better understand where a new hospital or service provider should be located. The development of these requirements may be an initial step in the application process and may make it possible to provide preferred metric tracking. Moreover, establishing operational guidelines may identify distinct applications (e.g., Microsoft Outlook) or legacy systems that contain important user information. Identifying these information sources may help a developer or integrator decide how and whether to interface an appointment tracking system with other information sources.

Components or software instances necessary to interface with other systems may be built using a variety of programming languages, depending on the systems in question. These components may facilitate the transfer of data to and from enterprise systems as application requirements dictate. Each integration component may access application programming interfaces (APIs) in order to access desired information instances. As application requirements change, an entity may enhance the integration components as needed.

Special conditional logic statements which may drive a rules engine in a server-based scheduling system may be created by the system administrator using a graphical user-interface with menu driven options. These rules may control how data that is applied to the domain is distributed to users in the domain.

At the beginning of a preferred data tracking process, a system administrator may add users to the system. As the domain evolves, the administrator may manage the users by adding more, modifying ones that currently exist, or removing ones that are no longer necessary.

Before a user interacts with the domain, the user may be linked to applications and privileges that they can use under their account, and have those applications and privileges made available to them. Each application may be a software instance that can be created, deployed and updated by developers that interface with the domain. Preferably, an application may be managed using the graphical user-interface provided by the computing system. This interface may be used to link applications to new users and unlink applications from users, as required for the consistent and efficient operation of the system. Once an application is linked to one or more users, changes to that application, including new deployments and updates, can be made available to the linked user.

For example, at 306, an appointment target may be added and managed. This step may be performed by various individuals including the target, an assistant with permission to act as the target, or an administrator. In operation, a target may access, create, and update availability calendars or data instances managed by the domain, and may have relevant transactions based on these instances re-distributed to other interested seekers and targets in the domain (as rules in the system dictate). The rules may be established by the target or by an administrator. In some cases, a set of targets may be established such that the set is treated collectively as an individual target, as indicated in FIG. 2 at 230. In other words, a group of targets may be treated collectively as a single target, with each target in the group sharing some set of assigned privileges. In practice, targets may be collected into groups so that they may share domain resources based on real-world affiliations, such as geographic location or job role.

At 308, a seeker may be added to and access the system in much the same way as a target. The seeker may also be linked to various applications or instances of data. For example, if the seeker is a patient, data instances may be linked to and distributed to the seeker in order to populate applications and forms with personal and medical information requested by the target doctor. In operation, a seeker may search for an available doctor based on several criteria including, for example, doctor name, location, time available, and specialty.

If a target satisfying a seeker's requirements has posted himself or herself as available to meet during a time that satisfies the seeker, the seeker may schedule an appointment at step 310. The appointment scheduling may result in a real time confirmed appointment as well as the triggering of several other operations. For example, at step 312, a notification may be sent to the target through any of several means including, for example, Email. In addition, at step 314, a target's schedule may be updated to reflect the newly scheduled meeting, and at step 316, reservations necessary (e.g., reserved space or telecommunication bandwidth) may be made.

As discussed above, step 304 may involve a hosting solution. Hosting may, itself, involve the steps of receiving a software platform that includes appointment tracking code and loading the software platform onto a server that is connected with a computer network, such as the Internet or an intranet, so that multiple users may access the software platform. Hosting may also include hosting services that accompany the hosting process. Payment, or other type of monetary value, for hosting the software platform may also be invoiced and collected—involving additional steps.

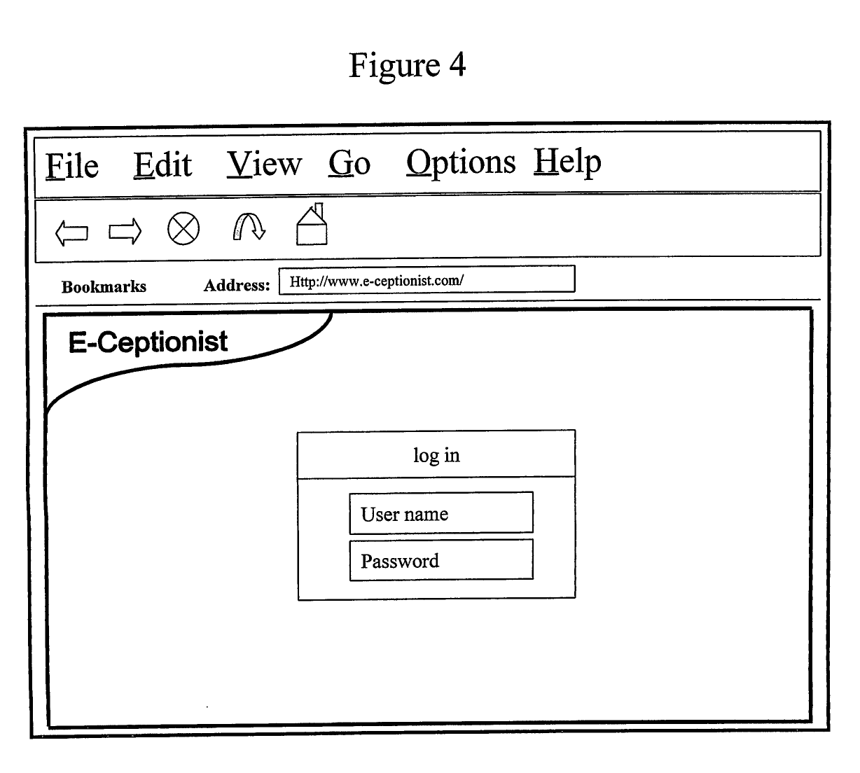

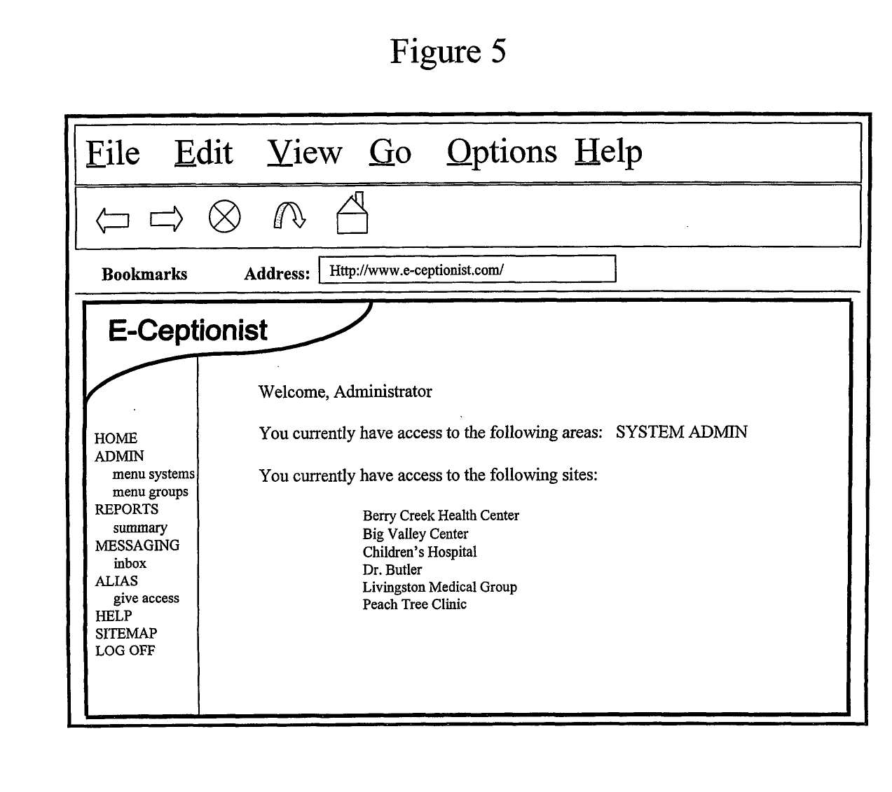

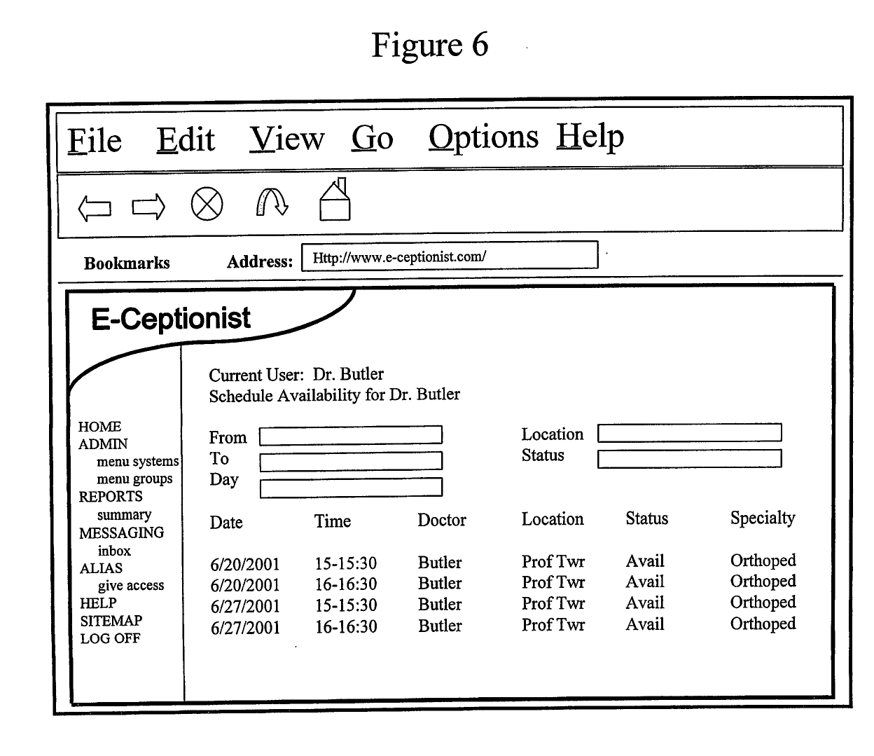

FIGS. 4 through 9 depict graphical user interfaces (GUIs) that may be presented during operation of a system like system 200 of FIG. 2. FIG. 4 shows one embodiment of a graphical user interface presented to a system user at log in. During log in, a user may be identified and preferences or privileges associated with the logged in user may be called. FIG. 5 shows one embodiment of a graphical user interface presented to a system administrator, and FIG. 6 shows one embodiment of a graphical user interface presented to an appointment target identifying blocks of available to meet time. In the GUI of FIG. 6, a target doctor, Dr. Butler, is establishing a clinic or making himself available for orthopedic services on Jun. 20 and 27, 2001. In the example of FIG. 6, Dr. Butler has made himself available for thirty minute meetings in the Professional Tower. In other embodiments, Dr. Butler could put more or less restrictions on her meeting availability.

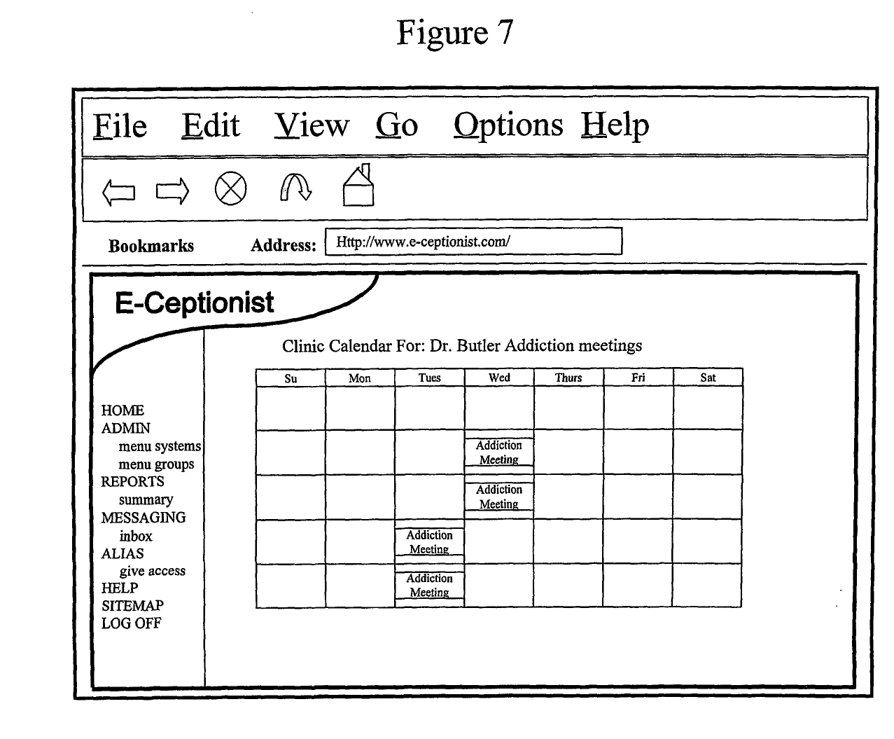

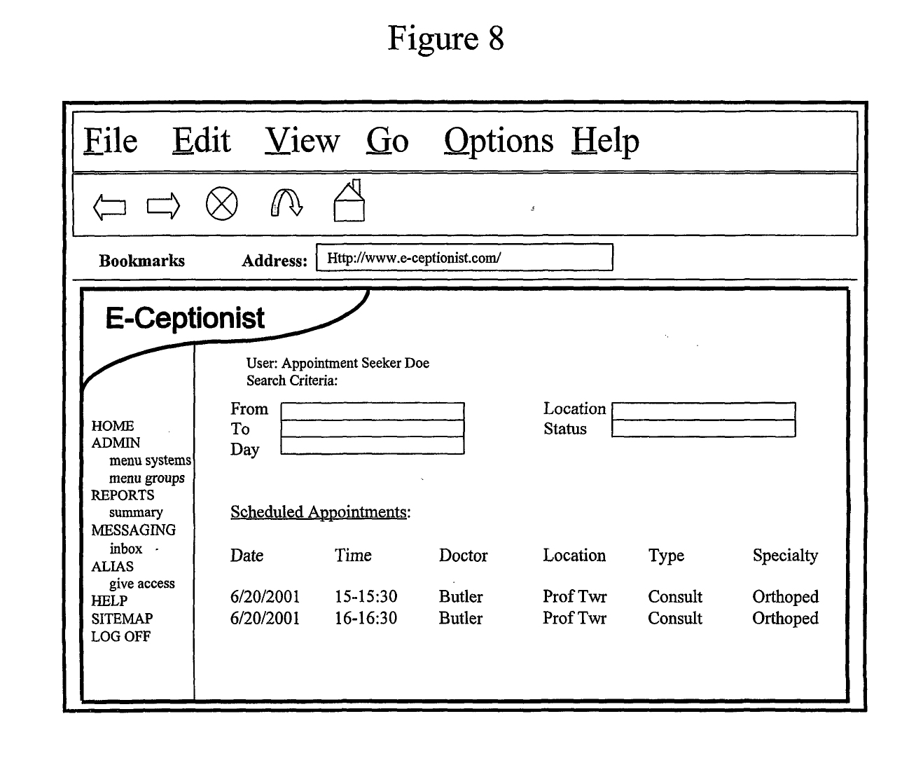

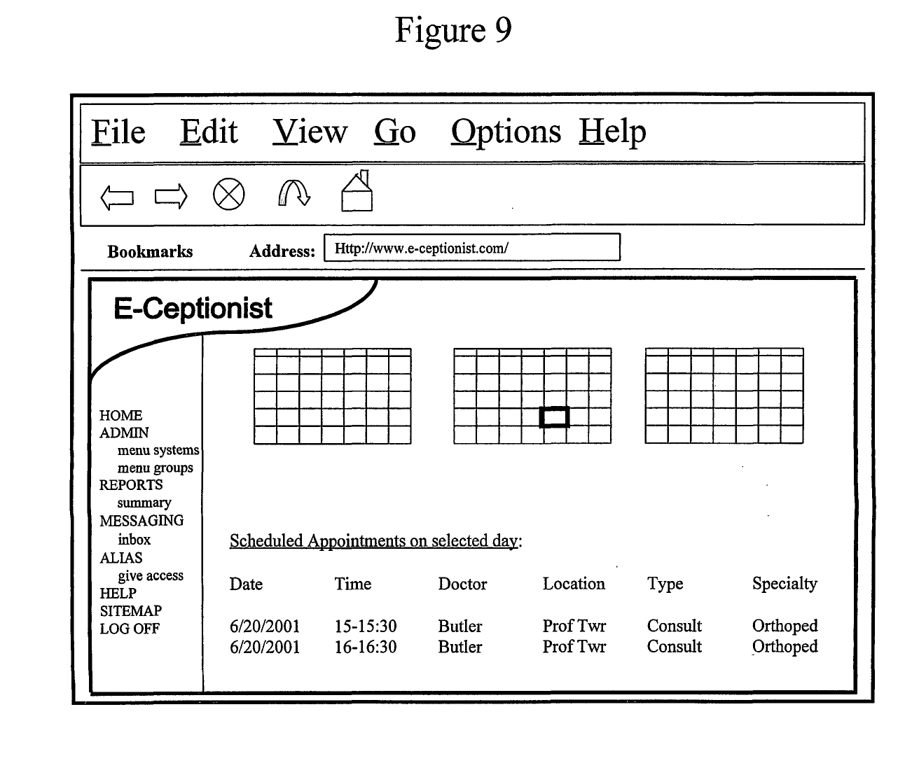

FIG. 7 shows one embodiment of a graphical user interface presented to an appointment seeker viewing a calendar of a specific target's available to meet time. As depicted, the calendar is for Dr. Butler's addiction meetings. FIG. 8 shows one embodiment of a graphical user interface presented to an appointment seeker searching for and scheduling an appointment. The appointment scheduled is with Dr. Butler, for orthopedic services in the Professional Tower. As depicted, the meeting is an initial consultation. FIG. 9 shows one embodiment of a graphical user interface presented to an appointment seeker depicting the seeker's scheduled appointments.

Though GUIs could be presented to users in any or all of several formats, FIGS. 4 through 9 depict GUIs as web pages in a browser window. The data entered in a web based scheduling system like system 200 may represent some real world object, place, thing, person, or collection and combination thereof. The entered data may be used to create a calendar, physical table or group of tables in a data store that will hold physical instances of those objects, places, things, or combinations, in records. Because in object oriented programming, class names in a data model may be used to instantiate physical tables in a data store, a developer may want to consider naming limitations of the particular database management system (DBMS).

Although the present invention has been described by way of detailed examples and illustrative embodiments, it should be understood that various changes, substitutions, and alterations could be made hereto without departing from the spirit and scope of the invention. Accordingly, the invention is not to be limited by the above detailed description, but rather is defined by the appended claims and equivalents thereof, to the maximum extent permissible by law.

What is claimed is:

1. A computer system for tracking appointment data between an appointment seeker and an appointment target, comprising:
a clinic engine operable to allow the appointment target to identify a first block of time as available to meet time;
an appointment engine operable to make available at least a portion of the first block of time to an appointment seeker in response to an appointment request;
an appointment seeker interface operable to allow the appointment seeker to schedule a first appointment during the first block of time; and
a notification engine operable to notify the appointment target of the first appointment.

2. The system of claim 1, wherein the clinic engine is further operable to allow the appointment target to identify allowable locations for the first appointment.

3. The system of claim 1, wherein the clinic engine is further operable to allow the appointment target to identify an allowable duration for the first appointment.

4. The system of claim 1, further comprising a server implementing the clinic engine, the appointment engine, and the notification engine.

5. The system of claim 1, further comprising:
an electronic mail interface operable to allow the notification engine to communicate via a distinct electronic mail application.

6. The system of claim 1, further comprising:
a legacy system interface operable to allow information about the appointment seeker located in a legacy system to be read and used to prepopulate forms associated with the computer system.

7. The system of claim 1, further comprising:
an update engine operable to modify the first block of time in response to a scheduled appointment such that a meeting time of the scheduled appointment is identified as unavailable to meet.

8. The system of claim 1, further comprising:
an update engine operable to modify the first block of time in response to a scheduled appointment such that a meeting time of the scheduled appointment is identified as unavailable to meet;
wherein the update engine, the clinic engine, the appointment engine, and the notification engine comprise web based applications.

9. A method for tracking appointment data, comprising:
maintaining a data store comprising data relating to an appointment schedule of an appointment target;
identifying an appointment opportunity for the appointment target as open for scheduling in response to an input;
receiving a request to schedule an appointment for the appointment opportunity;
granting the request; and
notifying the appointment target of the granted request.

10. The method of claim 9, further comprising:
identifying a second appointment opportunity for a second appointment target as open for scheduling in response to an second input;
receiving a second request to schedule a second appointment for the second appointment opportunity;
granting the second request; and
notifying the second appointment target of the second granted request.

11. The method of claim 9, further comprising:
modifying the data store to indicate the granted request.

12. The method of claim 9, wherein the data represents information relating to a type of appointment available for scheduling, a place available for scheduling, and a time limit for the appointment opportunity.

13. The method of claim 9, further comprising;
maintaining scheduling information relating to a type of appointment available for scheduling, a place available for scheduling, and a time limit for the appointment opportunity; and
reserving the place available for scheduling.

14. The method of claim 9, further comprising:
maintaining scheduling information relating to a type of appointment available, wherein the type comprises a telemedicine appointment.

15. The method of claim 9, further comprising:
maintaining scheduling information relating to a type of appointment available, wherein the type comprises a virtual appointment; and
reserving telecommunication services necessary for the virtual appointment.

16. The method of claim 9, further comprising:
prompting an appointment seeker to enter appropriate personal information.

17. The method of claim 9, further comprising:
prompting an appointment seeker to enter personal information;
comparing the personal information against stored information;
determining that the stored information comprises additional information about the appointment seeker; and
prepopulating segments of an appointment form with at least a portion of the additional information.

18. A computer system for tracking appointment data between a plurality of end users comprising:
an end user interface which may be accessible by a first end user of the system using standard communication protocols and which allows the first end user to preemptively indicate an availability window of the first end user;
a calendar engine operable to generate an active calendar for the first end user that indicates the availability window of the first end user;
a second end user interface which may be accessible to a second end user and which allows the second end user to access the active calendar and to select an appointment time from within the availability window; and
a scheduling engine that confirms the appointment time to the second end user without confirmation from the first end user

19. The system of claim 18, further comprising:
a notification engine operable to provide Email notification of the confirmed appointment time to the first end user; and
a update engine operable to modify the active calendar to indicate the unavailability of the appointment time to other end users.

20. A system for tracking appointment information, comprising:
a computer readable medium; and
an application stored on the computer readable medium, the application operable to:
maintain a data store comprising data relating to an appointment schedule of an appointment target;
identify an appointment opportunity for the appointment target as open for scheduling in response to an input;
receive a request to schedule an appointment for the appointment opportunity;
grant the request; and
notify the appointment target of the granted request.
